(19) United States
(12) Patent Application Publication
(10) Pub. No.: US 2019/0255131 A1
(43) Pub. Date: Aug. 22, 2019

(54) A COMPOSITION COMPRISING AN EXTRACT OF IRVINGIA GABONENSIS AND STRAINS OF BIFIDOBACTERIA FOR REGULATING AND REDUCING BODY WEIGHT

(71) Applicant: PROBIOTICAL S.P.A., Novara (IT)

(72) Inventor: Giovanni MOGNA, Novara (IT)

(21) Appl. No.: 16/343,329

(22) PCT Filed: Oct. 20, 2017
(86) PCT No.: PCT/IB2017/056522

§ 371 Date: Apr. 18, 2019

(30) Foreign Application Priority Data
Oct. 20, 2016 (IT) 102016000105831

(51) Int. Cl. A61K 36/22

(52) U.S. Cl. CPC A61K 36/22; A61P 3/04

(57) ABSTRACT

The present invention refers to a composition (C) comprising at least, or, alternatively, consisting of: (a) bacteria belonging to at least one strain of the genus Bifidobacterium and (b) a seed extract from seeds of the genus Irvingia, to said composition for use in treating obesity and to the utilization of this composition to favour body weight loss.

The present invention refers to a composition based on an extract of Irvingia and strains of Bifidobacteria for regulating and reducing body weight. The present invention also refers to a composition (C) comprising a mixture that comprises or, alternatively, consists of: (a) at least one bacterial strain belonging to the genus Bifidobacterium and (b) a seed extract from seeds of the genus Irvingia, preferably an Irvingia gabonensis extract. Moreover, the invention refers to said composition (C) for use in the treatment of obesity, and/or in treatment to favour/promote the regulation or reduction/loss of body weight.

Obesity is a disorder characterized by the excessive accumulation of body fat, which occurs according to different modalities based on gender and that exposes the subjects affected by it to various health risks, particularly regarding cardiovascular diseases, diabetes, obstructive sleep apnoea syndrome, ambulation problems, osteoarthrosis and several types of cancer.

In 1997, the World Health Organization (WHO) officially recognized obesity as a global epidemic and obesity currently represents the leading cause of preventable death worldwide, with the increase in prevalence in adults and children.

The WHO defines obesity through the Body Mass Index (BMI), a biometric measure that takes weight and height into account: subjects with a BMI greater than 30 kg/m² are considered to be obese, whereas individuals with a BMI in the range of 25 to 30 kg/m² are held to be overweight (“pre-obesity”).

According to said WHO definition, overweight subjects also have a greater risk of developing cardiovascular diseases and diabetes and, in Western societies, they tend to have lower self-esteem compared to persons whose BMI is considered to be “ideal”, and this can lead to depression or to other psychological and behavioural problems. The causes of excessive weight are principally overeating and hypometabolism, particularly the lack of an adequate amount of physical activity due to a sedentary lifestyle.

In the most severe cases, obesity is treated surgically. Currently, the most frequently prescribed pharmacological treatments are based on statins (e.g. tetralipstatin), which ensure only a modest reduction in body weight (2-3 kg/year more than control groups, see Padwal R et al, “Long-term pharmacotherapy for obesity and overweight”, in Raj S Padwal (edited by), Cochrane Database Syst Rev, no. 3, 2004, pp. CD004094) and they have adverse effects on the digestive tract, including steatorrhea.

In the case of severely obese subjects, amphetamines have been prescribed; amphetamines contribute to appetite suppression. However, their use is becoming increasingly more limited as they are psychotropic substances that can induce dependence.

It is apparent that the currently available treatments for treating obesity are thus invasive, often lead to unpleasant and serious side effects and can require several weeks to obtain significant and visible decreases in weight.

One of the principal problems is the problem of relapses at the end of treatment, which can lead to regaining all of the weight initially lost and in the severest cases even to a situation that is worse than the initial one (the so-called “yo-yo” effect).

Furthermore, when there are no appreciable results in the early stages of treatment, the subject can become unmotivated and drop out of the weight loss plan.

To respond to the limits of the prior art cited above, the present invention offers a new natural composition without side effects of any kind, comprising bacterial strains of one or more species belonging to the genus Bifidobacterium and a seed extract from seeds of the genus Irvingia, and which, taken orally, for example in the form of a tablet, enables rapid reduction/loss of excess body weight.

The object of the present invention is a composition (C) comprising a mixture that comprises or, alternatively, consists of:

at least one bacterial strain belonging to the genus Bifidobacterium, and

(b) a seed extract of a plant of the genus Irvingia, and

said composition further comprises, optionally,

(c) at least one ingredient or technological additive that is acceptable for pharmaceutical or food use,

with the exclusion of compositions in which Bifidobacterium longum BL04-DSM 23233 is the only bacterial strain of the species Bifidobacterium longum.

The object of the present invention is also a composition (C) as described above for reducing body weight in obese or overweight subjects by oral administration of (C).

A further object of the present invention is the use of the composition as described above in order to favour body weight loss in a subject, wherein said use does not have therapeutic purposes. For example, said use can be advantageous for subjects who intend to lose weight, for example for aesthetic or sports-related purposes, in the absence of an actual therapeutic necessity to lose weight.

Preferred embodiments shall be illustrated below, without the intention of limiting the scope and value in any manner.

Unless specified otherwise, the amount of a component in a composition refers to the percentage by weight of that component with respect to the total weight of the composition.

Unless specified otherwise, the indication that a composition “comprises” one or more components means that other components may be present in addition to the component or components specifically indicated and the indication that a composition “consists” of given components means that the presence of other components is excluded.

The composition (C) can consist of (a) and (b) or it can comprise other ingredients in addition to (a) and (b), which can be other active ingredients, bacteria belonging to genera other than Bifidobacterium or excipients such as those commonly used in the pharmaceutical and food sectors and for food supplements.

The composition of the present invention can be formulated in a solid, lyophilized or dried form, for example in powder or granules, or a liquid or semi-liquid form.

By way of non-limiting example, said excipients can be chosen from among the acceptable ingredients for pharmaceutical or food use that comprise all the auxiliary substances known to the expert in the field for the preparation of forms for oral administration such as diluents, absorbents, sweeteners, flavourings, colourings, lubricants, antiadherents, glidants, binders, disintegrants, surfactants, antimicrobials, antioxidants and substances capable of modifying the release of the active ingredient over time or of enabling the release thereof only under given physiological conditions, for example in a certain pH range.

The inventors have found that the association of specific probiotic bacteria and a seed extract of plants of the genus Irvingia promotes the loss of body weight in a surprisingly rapid manner and practically without significant side effects.

Preferably, in the composition (C) according to the present invention, the probiotic bacteria (a) belong to one to five strains, preferably two to four strains or three strains selected from among:
B. longum BL 04-DSM 23233,
B. longum DLBL 07-DSM 25669,
B. longum DLBL 08-DSM 25670,
B. longum DLBL 09-DSM 25671,
B. longum DLBL 10-DSM 25672,
B. longum DLBL 11-DSM 25673,

with the exclusion of compositions in which Bifidobacterium longum BL04-DSM 23233 is the only bacterial strain of the species Bifidobacterium longum.

More preferably, in the composition (C) according to the present invention, the probiotic bacteria (a) belong to a mixture containing each one of the following five strains:
B. longum DLBL 07-DSM 25669,
B. longum DLBL 08-DSM 25670,
B. longum DLBL 09-DSM 25671,
B. longum DLBL 10-DSM 25672,
B. longum DLBL 11-DSM 25673,

optionally in association with the strain Bifidobacterium longum BL04-DSM 23233.

Said mixture (a) of bacterial strains preferably consists of: B. longum DLBL 07-DSM 25669, B. longum DLBL 08-DSM 25670, B. longum DLBL 09-DSM 25671, B. longum DLBL 10-DSM 25672 and B. longum DLBL 11-DSM 25673.

It has surprisingly been found that the administration of a composition comprising an extract of Irvingia with the combination of probiotic bacteria belonging to said 5 strains (that is, with a mixture of probiotic bacteria in which each one of said 5 strains is present) resulted in a weight loss even greater than 1 kg per week in obese or overweight subjects.

This loss is greater than that obtained following administration of similar compositions in which not all 5 strains were present.

In a preferred embodiment of the invention, in the composition (C), said strain of probiotic bacteria (a) comprises at least one strain belonging to the species Bifidobacterium breve chosen from the group comprising or, alternatively, consisting of:
Bifidobacterium breve BR03-DSM 16604, deposited on Jul. 20, 2004 under the name of Probiotical SpA,
Bifidobacterium breve BR04-DSM 16596, deposited on Jul. 21, 2004 under the name of Probiotical SpA,
Bifidobacterium breve DSM 20213, preferably in which said probiotic bacterial strain (a) further comprises at least one bacterial strain belonging to the species Bifidobacterium longum, preferably the bacterial strain Bifidobacterium longum BL04, deposited with the DSMZ on Jan. 12, 2010 under accession number DSM 23233, with the exclusion of compositions in which Bifidobacterium longum BL0 4-DSM 23233 is the only bacterial strain of the species Bifidobacterium longum and Bifidobacterium breve BR03-DSM 16604 is the only bacterial strain of the species Bifidobacterium breve.

Irvingia gabonensis is a plant native to West Africa and it has been traditionally used for food purposes. Its inhibiting action on the fatty tissue formation mechanism starting from undifferentiated mesenchyme cells (adipogenesis) is reported in Oben, J. E., et al. Lipids Health Dis. 2008 Nov. 13; 7:44.

The inventors have surprisingly found that the administration of a composition comprising (a) probiotic bacteria belonging to the genus Bifidobacterium and (b) an Irvingia gabonensis seed extract to obese or overweight subjects results in a weight loss greater than the weight loss that can be obtained through the administration of Irvingia gabonensis seed extract alone.

Moreover, subjects who have orally taken the association of probiotic bacteria (a) and the extract (b) as defined above have not reported any side effects, while headache, flatulence and difficulty sleeping have been indicated as adverse effects due to the use of Irvingia gabonensis, in Egras, A. M. et al. J Obes. 2011. pii: 297315.

In a preferred embodiment of the invention, said seed extract of a plant of the genus Irvingia (b) comprises a seed extract of the species Irvingia gabonensis, more preferably said extract is a dry extract of Mangifera Indica subsp. Gabonensis (such as IGOB131™).

In a preferred embodiment of the invention, the composition (C) comprises an extract (b) of Irvingia gabonensis standardized in polyphenols and alkaloids. Preferably, (b) comprises no less than 40% by weight of polyphenols, with respect to the total weight of (b), said polyphenols being quantified by UV spectroscopy at 575 nm and/or no less than 1.5% by weight of polyphenols, with respect to the total weight of (b), said polyphenols being quantified by UV spectroscopy at 760 nm, and no less than 1% by weight of alkaloids, with respect to the total weight of (b), said alkaloids being quantified by UV spectroscopy at 575 nm.

In a preferred embodiment of the invention, in the composition (C), the ratio by weight between (a) and (b) in said mixture is comprised from 1:10 to 10:1, preferably from 1:5 to 5:1, even more preferably from 1:3 to 3:1, for example 1:1.

In the composition (C) according to the present invention, the bacterial strains belonging to the species Bifidobacterium are present at a concentration comprised from 1×10⁶ to 1×10¹¹, preferably from 1×10⁷ to 1×10¹⁰, even more preferably from 1×10⁸ to 1×10⁹ CFU/g referring to the total weight of (C).

The following compositions represent preferred embodiments of the invention:

Composition 1

Bacteria:
B. longum DLBL 07-DSM 25669,
B. longum DLBL 08-DSM 25670,
B. longum DLBL 09-DSM 25671,
B. longum DLBL 10-DSM 25672,
B. longum DLBL 11-DSM 25673, or

(b) Seed extract of Irvingia gabonensis subsp. Gabonensis.

Composition 2

(a) Bacteria:
B. longum DLBL 07-DSM 25669,
B. longum DLBL 08-DSM 25670,
B. longum DLBL 09-DSM 25671,
B. longum DLBL 10-DSM 25672,
B. longum DLBL 11-DSM 25673, or
B. breve BR03-DSM 16604;

(b) Seed extract of Irvingia gabonensis subsp. Gabonensis.

Composition 3

(a) Bacteria:
B. longum BL 04-DSM 23233,
B. longum DLBL 07-DSM 25669,
B. longum DLBL 08-DSM 25670,
B. longum DLBL 09-DSM 25671,
B. longum DLBL 10-DSM 25672,
B. longum DLBL 11-DSM 25673;

(b) Seed extract of Irvingia gabonensis subsp. Gabonensis.

Composition 4

(a) Bacteria:
B. longum BL 04-DSM 23233,
B. longum DLBL 07-DSM 25669,
B. longum DLBL 08-DSM 25670,
B. longum DLBL 09-DSM 25671,
B. longum DLBL 10-DSM 25672,
B. longum DLBL 11-DSM 25673, or
B. breve BR03-DSM 16604;

(b) Seed extract of Irvingia gabonensis subsp. Gabonensis.

In one embodiment, the present invention provides the composition (C) as described above for use in reducing body weight in obese or overweight subjects by oral administration of (C).

In a preferred embodiment, the present invention provides the composition (C) for said use, wherein the reduction in body weight is equal to at least 1 kg for each week of treatment.

In one embodiment, the present invention concerns the use of the composition (C) as defined above in order to favour body weight loss in a subject, wherein said use does not have therapeutic purposes. Said composition (C) can be used for the therapeutic purposes of weight loss in non-obese and non-overweight subjects, according to the definition of the World Health Organization reported above, for example (non-limiting) for aesthetic purposes or to improve sports performances or a subject's physical fitness in general.

In one embodiment, the present invention provides a medical device in the form of a preparation for oral intake comprising the composition (C) as described above.

In the context of the present invention, the term “medical device” is used in accordance with meaning provide in the Italian legislative decree no. 46 of Feb. 24, 1997, that is, it indicates a substance or another product used alone or in combination, intended by the manufacturer to be used in humans for the purpose of diagnosis, prevention, monitoring, therapy or alleviation of disease, and which does not achieve its principal intended action in or on the human body by pharmacological or immunological means, or by means of a metabolic process, but which may be assisted in its function by such means.

As a non-limiting example, this medical device may be in the form of a capsule, tablet, powder that is at least partially oral soluble or water soluble, granules, pellets, microparticles, optionally contained in a sachet or in a capsule or in a mini-tablet, a liquid or semi-solid preparation, a gel, a suspension, a solution, a biphasic liquid system and equivalent forms.

Preferably, the medical device according to the invention is in the form of a sachet containing powder that is at least partially water soluble.

In one embodiment, the present invention offers the use of said medical device as an adjuvant to lose body weight and for weight control through the loss of body fat mass and a percentage increase in non-adipose body mass.

The following examples represent typical embodiments of the present invention, without limiting the scope thereof.

### EXAMPLE 1

The composition (C) according to the present invention can comprise:

Composition 1

(a) Bacteria:
B. longum DLBL 07-DSM 25669,
B. longum DLBL 08-DSM 25670,
B. longum DLBL 09-DSM 25671,
B. longum DLBL 10-DSM 25672,
B. longum DLBL 11-DSM 25673,

(b) Seed extract of Irvingia gabonensis subsp. Gabonensis.

Composition 2

(a) Bacteria:
B. longum DLBL 07-DSM 25669,
B. longum DLBL 08-DSM 25670,
B. longum DLBL 09-DSM 25671,
B. longum DLBL 10-DSM 25672,
B. longum DLBL 11-DSM 25673,
B. breve BR03-DSM 16604;

(b) Seed extract of Irvingia gabonensis subsp. Gabonensis.

Composition 3

(a) Bacteria:
B. longum BL 04-DSM 23233,
B. longum DLBL 07-DSM 25669,
B. longum DLBL 08-DSM 25670,
B. longum DLBL 09-DSM 25671,
B. longum DLBL 10-DSM 25672,
B. longum DLBL 11-DSM 25673;

(b) Seed extract of Irvingia gabonensis subsp. Gabonensis.

Composition 4

(a) Bacteria:
B. longum BL 04-DSM 23233,
B. longum DLBL 07-DSM 25669,
B. longum DLBL 08-DSM 25670,
B. longum DLBL 09-DSM 25671,
B. longum DLBL 10-DSM 25672,
B. longum DLBL 11-DSM 25673,
B. breve BR03-DSM 16604;

(b) Seed extract of Irvingia gabonensis subsp. Gabonensis.

### EXAMPLE 2

The composition (C) according to the invention (Example 1, similar results were obtained with each one of the compositions 1-4) was administered for 10 weeks to obese or overweight persons (n=7, 3 females, 4 males, BMI ranging between 26 and 40 kg/m²).

The data obtained (on average) after 10 weeks of treatment (one dose per day) are as follows:
Reduction in weight: 13.4 kg (on average, 1.34 kg/week)
Reduction in waistline: 13.cm kg (on average, 1.36 kg/week)
Reduction in fat percentage: 9%
Reduction in total cholesterol (5 subjects): 53 mg/dl.

Reduction in glycaemia (5 subjects): 19.5 mg/dl. None of the subjects reported side effects relating to the treatment with the composition according to the invention.

Complete data concerning each patient treated are reported in Tables 1-7.

By comparison (source: Ngondi, J. L. et al. Lipids Health Dis. 2009, 8:7), the administration of the extract (b) alone, without probiotic bacteria (a) for 10 weeks (600 mg/day) results in a weight decrease of 12.8 kg on average (12.1 kg with respect to the placebo), and in a lowering of total cholesterolaemia by 39.8 mg/dl (37 with respect to the placebo).

The composition according to the invention thus demonstrates a greater capacity for weight reduction and for lowering cholesterolaemia compared to compositions comprising only the Irvingia gabonensis extract and it is free of side effects.

TABLE 1
Patient 1
Age at test entry 56.44
Gender Male

| | Pre-test | | | | |
|---|---|---|---|---|---|
| Variables | data | Week 4 | Week 8 | Week 10 | Difference |
| Weight (kg) | 96 | 88 | 85 | 84 | 12 |
| Waistline (cm) | 110 | 102 | 98 | 96 | 14 |
| Leptin (ng/ml) | | | | | 0 |
| Adipose tissue (%) | 45 | 40 | 37 | 35 | 10 |
| LDL cholesterol | | | | | 0 |
| (mg/dl) | | | | | |
| Total cholesterol | 181 | 135 | 112 | 120 | 61 |
| (mg/dl) | | | | | |
| C-reactive protein | | | | | 0 |
| (mg/l) | | | | | |
| Glucose (mg/dl) | | | | | 0 |

TABLE 2
Patient 2
Age at test entry 56.00
Gender Male

| | Pre-test | | | | |
|---|---|---|---|---|---|
| Variables | data | Week 4 | Week 8 | Week 10 | Difference |
| Weight (kg) | 122 | 119 | 115 | 108 | 14 |
| Waistline (cm) | 123 | 117 | 115 | 111 | 12 |
| Leptin (ng/ml) | | | | | 0 |
| Adipose tissue (%) | 49 | 45 | 43 | 42 | 7 |
| LDL cholesterol | | | | | 0 |
| (mg/dl) | | | | | |
| Total cholesterol | 198 | | | 192 | 6 |
| (mg/dl) | | | | | |
| C-reactive protein | 3.3 | | | 1.7 | 1.6 |
| (mg/l) | | | | | |
| Glucose (mg/dl) | 118 | 106 | 98 | 96 | 22 |

TABLE 3
Patient 3
Age at test entry 53.00
Gender Male

| | Pre-test | | | | |
|---|---|---|---|---|---|
| Variables | data | Week 4 | Week 8 | Week 10 | Difference |
| Weight (kg) | 89 | 85 | 83 | 77 | 12 |
| Waistline (cm) | 97 | 94 | 92 | 89 | 8 |
| Leptin (ng/ml) | | | | | 0 |
| Adipose tissue (%) | 46 | 42 | 40 | 36 | 10 |
| LDL cholesterol | 67 | | | | 67 |
| (mg/dl) | | | | | |
| Total cholesterol | 160 | | | | 160 |
| (mg/dl) | | | | | |
| C-reactive protein | 3 | | | 1.6 | 1.4 |
| (mg/l) | | | | | |
| Glucose (mg/dl) | 92 | 94 | 88 | 89 | 3 |

TABLE 4
Patient 4
Age at test entry 46.00
Gender Male

| | Pre-test | | | | |
|---|---|---|---|---|---|
| Variables | data | Week 4 | Week 8 | Week 10 | Difference |
| Weight (kg) | 93 | 89 | 86 | 79 | 14 |
| Waistline (cm) | 105 | 99 | 97 | 90 | 15 |
| Leptin (ng/ml) | | | | | 0 |
| Adipose tissue (%) | 47 | 44 | 42 | 36 | 11 |
| LDL cholesterol | | | | | 0 |
| (mg/dl) | | | | | |
| Total cholesterol | 195 | 172 | 163 | 130 | 65 |
| (mg/dl) | | | | | |
| C-reactive protein | 3.7 | | 2.1 | 1.4 | 2.3 |
| (mg/l) | | | | | |
| Glucose (mg/dl) | 115 | | 97 | 86 | 29 |

TABLE 5
Patient 5
Age at test entry 45.00
Gender Male

| | Pre-test | | | | |
|---|---|---|---|---|---|
| Variables | data | Week 4 | Week 8 | Week 10 | Difference |
| Weight (kg) | 112 | 107 | 101 | 97 | 15 |
| Waistline (cm) | 118 | 110 | 103 | 99 | 19 |
| Leptin (ng/ml) | | | | | 0 |
| Adipose tissue (%) | 49 | 46 | 44 | 41 | 8 |
| LDL cholesterol | | | | | 0 |
| (mg/dl) | | | | | |
| Total cholesterol | 230 | | | 155 | 75 |
| (mg/dl) | | | | | |
| C-reactive protein | 4.1 | | | 1.8 | 2.3 |
| (mg/l) | | | | | |
| Glucose (mg/dl) | 124 | | | 95 | 29 |

TABLE 6
Patient 6
Age at test entry 62.00
Gender Male

| | Pre-test | | | | |
|---|---|---|---|---|---|
| Variables | data | Week 4 | Week 8 | Week 10 | Difference |
| Weight (kg) | 85 | 80 | 77 | 73 | 12 |
| Waistline (cm) | 99 | 95 | 91 | 85 | 14 |
| Leptin (ng/ml) | | | | | 0 |
| Adipose tissue (%) | 37 | | 33 | 30 | 7 |
| LDL cholesterol | | | | | 0 |
| (mg/dl) | | | | | |
| Total cholesterol | | | | | 0 |
| (mg/dl) | | | | | |
| C-reactive protein | | | | | 0 |
| (mg/l) | | | | | |
| Glucose (mg/dl) | 99 | | | 87 | 12 |

TABLE 7
Patient 7
Age at test entry 34.00
Gender Male

| | Pre-test | | | | |
|---|---|---|---|---|---|
| Variables | data | Week 4 | Week 8 | Week 10 | Difference |
| Weight (kg) | 97 | 92 | 88 | 82 | 15 |
| Waistline (cm) | 102 | 98 | 93 | 89 | 13 |
| Leptin (ng/ml) | | | | | 0 |
| Adipose tissue (%) | 41 | 39 | 36 | 31 | 10 |
| LDL cholesterol | | | | | 0 |
| (mg/dl) | | | | | |
| Total cholesterol | 220 | | | 163 | 57 |
| (mg/dl) | | | | | |
| C-reactive protein | | | | | 0 |
| (mg/l) | | | | | |
| Glucose (mg/dl) | 113 | | | 91 | 22 |

1. A composition (C) comprising a mixture which comprises or, alternatively, consists of:
(a) at least one bacterial strain belonging to the genus Bifidobacterium, and
(b) a seed extract of a plant of the genus Irvingia, preferably wherein said seed extract of a plant of the genus Irvingia (b) comprises a seed extract of the species Irvingia gabonensis, more preferably said extract is a dry extract of Mangifera Indica subsp. Gabonensis; and
said composition further comprises, optionally,
(c) at least one ingredient or technological additive that is acceptable for pharmaceutical or food use, with the exclusion of compositions in which Bifidobacterium longum BL04-DSM 23233 is the only bacterial strain of the species Bifidobacterium longum.

2. The composition (C) according to claim 1, wherein, in said mixture, (a) comprises or, alternatively, consists of at least one probiotic bacterial strain belonging to the species Bifidobacterium longum, or at least one probiotic bacterial strain belonging to the species Bifidobacterium breve.

3. The composition (C) according to claim 1, wherein, in said mixture, (a) comprises or, alternatively, consists of at least two different bacterial strains, at least one of which belongs to the species Bifidobacterium longum (including the bacterial strain Bifidobacterium longum BL04-DSM 23233) and/or at least one belongs to the species Bifidobacterium breve.

4. The composition (C) according to claim 3, wherein, in said mixture, (a) comprises or, alternatively, consists of: at least two bacterial strains belonging to the species Bifidobacterium longum, including the bacterial strain Bifidobacterium longum BL04-DSM 23233; or at least two bacterial strains belonging to the species Bifidobacterium breve; or at least one bacterial strain belonging to the species Bifidobacterium longum, including the bacterial strain Bifidobacterium longum BL04-DSM 23233, and at least one bacterial strain belonging to the species Bifidobacterium breve.

5. The composition (C) according to any one of the preceding claims, wherein said probiotic bacterial strain (a) belonging to the species Bifidobacterium longum is selected from the group comprising or, alternatively, consisting of:
B. longum BL 04-DSM 23233,
B. longum DLBL 07-DSM 25669,
B. longum DLBL 08-DSM 25670,
B. longum DLBL 09-DSM 25671,
B. longum DLBL 10-DSM 25672,
B. longum DLBL 11-DSM 25673, or
mixtures of bacterial strains thereof;
preferably, said mixture of bacterial strains consists of: B. longum DLBL 07-DSM 25669, B. longum DLBL 08-DSM 25670, B. longum DLBL 09-DSM 25671, B. longum DLBL 10-DSM 25672 and B. longum DLBL 11-DSM 25673.

6. The composition (C) according to any one of the preceding claims, wherein said probiotic bacterial strain (a) comprises at least one strain belonging to the species Bifidobacterium breve and is selected from the group comprising or, alternatively, consisting of:
Bifidobacterium breve BR03-DSM 16604, deposited on Jul. 20, 2004 under the name of Probiotical SpA,
Bifidobacterium breve BR04-DSM 16596, deposited on Jul. 21, 2004 under the name of Probiotical SpA,
Bifidobacterium breve DSM 20213, preferably wherein said probiotic bacterial strain (a) further comprises at least one bacterial strain belonging to the species Bifidobacterium longum, preferably the bacterial strain Bifidobacterium longum BL04, deposited with the DSMZ on Jan. 12, 2010 under accession number DSM 23233.

7. The composition (C) according to any one of the preceding claims, wherein the ratio by weight between (a) and (b) in said mixture is comprised from 1:10 to 10:1, preferably from 1:5 to 5:1, even more preferably from 1:3 to 3:1, for example 1:1.

8. The composition (C) according to at least one of the preceding claims, for use in reducing body weight in obese or overweight subjects by oral administration of (C).

9. A use of the composition (C) according to at least one of claims 1-6 in order to favour body weight loss in a subject, wherein said use does not have therapeutic purposes.

10. A medical device in the form of a preparation for oral intake comprising the composition (C) according to at least one of claims 1-7, comprising at least the following ingredients:
Composition 1
(a) Bacteria:
B. longum DLBL 07-DSM 25669,
B. longum DLBL 08-DSM 25670,
B. longum DLBL 09-DSM 25671,
B. longum DLBL 10-DSM 25672,
B. longum DLBL 11-DSM 25673,
(b) Seed extract of Irvingia gabonensis subsp. Gabonensis; or
composition 2:
(a) Bacteria:
B. longum DLBL 07-DSM 25669,
B. longum DLBL 08-DSM 25670,
B. longum DLBL 09-DSM 25671,
B. longum DLBL 10-DSM 25672,
B. longum DLBL 11-DSM 25673,
B. breve BR03-DSM 16604;
(b) Seed extract of Irvingia gabonensis subsp. Gabonensis; or
composition 3
(a) Bacteria:
B. longum BL 04-DSM 23233,
B. longum DLBL 07-DSM 25669,
B. longum DLBL 08-DSM 25670,
B. longum DLBL 09-DSM 25671,
B. longum DLBL 10-DSM 25672,
B. longum DLBL 11-DSM 25673;
(b) Seed extract of Irvingia gabonensis subsp. Gabonensis; or
composition 4
(a) Bacteria:
B. longum BL 04-DSM 23233,
B. longum DLBL 07-DSM 25669,
B. longum DLBL 08-DSM 25670,
B. longum DLBL 09-DSM 25671,
B. longum DLBL 10-DSM 25672,
B. longum DLBL 11-DSM 25673,
B. breve BR03-DSM 16604,
(b) Seed extract of Irvingia gabonensis subsp. Gabonensis.
